(19) United States
(12) Patent Application Publication
(10) Pub. No.: US 2023/0040142 A1
(43) Pub. Date: Feb. 9, 2023

(54) MOLDED COMPOSITION FOR TOPICAL APPLICATION AND USES THEREOF

(71) Applicant: Fischer Pharmaceuticals LTD, Bnei Brak (IL)

(72) Inventors: Tal Leizer, Zikhron Ya'akov (IL); Danny Ditzer, Rishon LeZion (IL); Sharon Gertler, Ra'anana (IL); Nurit Harel, Tel Aviv (IL)

(21) Appl. No.: 17/790,351

(22) PCT Filed: Dec. 30, 2020
(86) PCT No.: PCT/IL2020/051362

§ 371 Date: Jun. 30, 2022

(30) Foreign Application Priority Data
Dec. 30, 2019 (IL) 271766

(51) Int. Cl. A61K 8/73; A61K 8/04; A61K 8/36; A61K 8/02; A61Q 19/00

(52) U.S. Cl. CPC A61K 8/73; A61K 8/042; A61K 8/361; A61K 8/0216; A61Q 19/007

(57) ABSTRACT

The present disclosure provides solid molded formulations comprising an emulsion gel comprising a gel matrix, water and lipophilic droplets comprising one or more emulsifiers and lipophilic material, the lipophilic droplets being dispersed within the gel matrix, wherein the gel matrix is formed of at least one gel forming material that is water insoluble at a temperature below 40° C.; the at least one gel forming material is present at a concentration higher than 1% by weight out of the total weight of the solid molded composition; the lipophilic material is present at a concentration of at least 5% by weight out of the total weight of the solid molded composition; at least 80% of the emulsifiers are solid at room temperature; and the said at least one gel forming material, one or more emulsifiers, lipophilic material and water provide together said solid molded composition with a discrete shape that is deformable upon shearing against a surface. Also disclosed are methods of producing the formulations.

### TECHNOLOGICAL FIELD

This invention generally relates to topical formulations.

### BACKGROUND ART

References considered to be relevant as background to the presently disclosed subject matter are listed below:

Chinese patent application publication No. CN108158832

U.S. Pat. No. 9,125,839

U.S. Pat. No. 9,878,036

U.S. Pat. No. 6,319,507
Farjami, T., Madadlou, A., An overview on preparation of emulsion-filled gels and emulsion particulate gels, Trends in Food Science & Technology 86:85-94 (2019).
Gohtani, S., Kim, K.-H., & Yamano, Y. (2000). Hydrocolloids, 91-96. Rheological properties and microstructure of monodispersed O/W emulsion agar gel.

Acknowledgement of the above references herein is not to be inferred as meaning that these are in any way relevant to the patentability of the presently disclosed subject matter.

### BACKGROUND

One of the issues with known skin delivery forms such as creams, ointments, serum, solutions, gels and lotions is the inconsistent amounts of active ingredients applied onto the skin. Many such actives tend to cause an undesired effect when the applied amount or the local concentration of the actives is too low or too high. Also cost-wise, one may want to apply a precise amount of the product.

In recent years, gels have been used for topical delivery. In general, gels can be differentiated into two types, according to the properties of their liquid phase: organogels (or oleogels) which contains an organic (or oily) liquid phase, and hydrogels which contain water as their liquid external phase.

Gels may also be used in combination with particulate matter embedded therein.

For example, CN108158832A describes a matrix composition comprising the matrix and grease particles dispersed in the matrix. The matrix is a solid or semisolid product, and the grease particles contain one or a plurality of humectants. The matrix can be in a form of a gel, e.g. hydrogel.

In addition, U.S. Pat. No. 9,125,839 describes a composition for external use on skin that contains 0.1% by mass to 10% by mass of a component (A) being a water-soluble polymer obtained by mixing agar with xanthan gum, 0.5% by mass to 40% by mass of a divalent polyol as a component (B); moisturizers as a component (C) and 30% by mass or more of a component (D) being water; and oil as a component (E).

Further, U.S. Pat. No. 9,878,036 describes a pharmaceutical composition comprising a water-soluble polymer matrix in which are dispersed droplets of oil, the composition comprising at least one immunomodulator selected from an adjuvant, an antigen or a combination thereof. The pharmaceutical composition is typically formulated for oral administration.

U.S. Pat. No. 6,319,507 describes crushable gel beads formed of an agar complex as a cosmetic or pharmaceutical, etc. delivery vehicle for topical delivery of biologically or cosmetically active agents. The beads are described as complexes of a continuous phase of agar gel in a self-supporting solid or semi-solid form with a restraining polymer.

Farjami, T., Madadlou, A (2019) describe the preparation of emulsion filled gels and emulsion particulate gels, both being a class of soft solid like materials. These composite materials are describes as either a polymeric gel matrix into which emulsion droplets are incorporated (emulsion-filled gels), or a network of aggregated emulsion droplets (emulsion particulate gels).

U.S. Pat. No. 5,540,921 describes a solid oil in water (o/w) cosmetic composition, a process for molding it and a container used for aqueous type solid cosmetics.

US 2011/0306679 describes a composition that has (a) at least one thermo-reversible polysaccharide chosen from agar; (b) at least one softening agent chosen from a cationic surfactant, an anionic surfactant, a nonionic esterified sugar surfactant, a polyorganosiloxane-containing polymer, a sugar silicone surfactant, and mixtures thereof; (c) at least one oil; and (d) water.

Gohtani, S., Kim (2000) describes the effect of oil droplets on the rheological properties and microstructure of monodispersed O/W emulsion agar gel using 1% agar.

Additional publications describing formulations including a gel component are US Patent application publication No. 20180263866; Chinese patent application publication No. CN105213318; U.S. Pat. No. 8,586,078; European Patent No. 1176941; U.S. Pat. No. 6,204,308; International Patent Application No. WO2000023047; European Patent No. 1030642; U.S. Pat. No. 5,674,504; US Patent Application Publication No. 20170367937; U.S. Pat. No. 9,808,429; European Patent No. 1942908; U.S. Pat. Nos. 7,993,677; 6,979,467; 6,090,373; 5,733,531; International Patent Application No. WO2016094617; International

Patent Application No. WO2019088056; and International Patent Application No. WO2017164429.

### GENERAL DESCRIPTION

The present disclosure provides, in accordance with a first of its aspects, a solid spreadable composition, referred to herein, at times, by the term solid molded composition, or solid mold, comprising an emulsion gel including a gel matrix, water and lipophilic droplets dispersed within the gel matrix, the composition further comprising one or more emulsifiers and lipophilic material,

wherein

the gel matrix is formed of at least one gel forming material (this includes one or more gel forming agents) that is water insoluble at a temperature below 40° C.;

the at least one gel forming material is present at a concentration higher than 1% by weight out of the total weight of the solid molded composition;

the lipophilic material is present at a concentration of at least 5% by weight out of the total weight of the solid molded composition;

at least 80% of the emulsifiers are solid at room temperature (25° C.±2° C.); and

the at least one gel forming material, one or more emulsifiers, lipophilic material and water, forming together said composition, have a solid discrete shape that is deformable upon shearing against a surface.

In some aspects, there is provided a solid molded composition, comprising an emulsion gel including a gel matrix, water, and lipophilic droplets dispersed within the gel matrix, the composition is further comprising one or more emulsifiers and lipophilic material

wherein

the gel matrix is formed of at least one gel-forming material, wherein the gel-forming material comprises agarose (e.g. agar) at a concentration higher than 1% by weight out of the total weight of the solid molded composition, and at times at least and up to 1.8%);

the lipophilic material is present at a concentration of at least 5% by weight out of the total weight of the solid molded composition;

at least 80% of the emulsifiers are solid at room temperature (25° C.±2° C.); and

the at least one gel forming material, one or more emulsifiers, lipophilic material and water, forming together said composition, have a solid discrete shape that is deformable upon shearing against a surface.

Also provided by the present disclosure is a method of producing the solid molded composition disclosed herein, the method comprises
introducing into a hydrophilic phase that comprises, water and at least one gel forming agent, a lipophilic phase comprising lipophilic material and one or more emulsifiers, said introducing comprises stirring of the hydrophilic phase and the lipophilic phase while said hydrophilic phase is at a temperature of at least 70° C. and the lipophilic phase is at a temperature of at least 60° C., at times at least 70° C., until a homogenized emulsion gel is formed;
adjusting pH of the emulsion gel to a pH 4-9; at times 6-8.5 and
molding the emulsion gel;

wherein

the at least one gel forming material that is water insoluble at a temperature below 40° C. and/or comprises agarose;

the lipophilic material is present in an amount to constitutes at least 5% by weight out of the total weight of the solid molded composition;

the at least one gel forming material is present in an amount which is higher than 1% by weight out of the total weight of the solid molded composition;

at least 80% of said one or more emulsifiers are solid at room temperature (25° C.±2° C.); and

the said at least one gel forming material, one or more emulsifiers, lipophilic material and water provide together a solid molded composition with a discrete shape that is deformable upon shearing against a surface.

Further provided herein is a package comprising one or a plurality of discrete solid molded compositions disclosed herein. The package can include a single molded composition or a plurality of separable solid molded compositions.

Yet further, disclosed herein is a method for topical application of the molded composition onto a bodily portion (as defined hereinbelow, e.g. skin), the method comprises shearing at least one of said molded compositions against the bodily portion. A unique feature of the molded composition is that it does not leave any residues on the body once it is spread against the body surface, as further discussed below.

### DESCRIPTION OF DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1E are images of solid deformable molds of different colors or sizes and forms, comprising the topical spreadable formulation in accordance with examples of the present disclosure, with FIG. 1A showing solid molds having 1.5% w/w Agar-Agar in accordance with one example of the present disclosure; FIG. 1B is an image of a sun-screen solid formulation comprising 2% w/w of Agar-Agar, and FIG. 1C is a hand cream formulation comprising 2% w/w of Agar-Agar; FIG. 1D is an image providing exemplary dimensions of the solid formulations; while FIG. 1E is an image of a package comprising a plurality of solid molded compositions.

FIG. 2 is an image of a solid mold having 1.2% w/w of Agar-Agar, in accordance with one example of the present disclosure.

FIG. 3 is an image of a Comparative Example where a mold is prepared from a composition comprising 1.0% w/w of Agar-Agar.

FIG. 4 is an image of a Comparative Example after being spread onto a subject's skin, the mold comprising 2.5% w/w of Agar-Agar.

FIGS. 5A-5B are images of the molded composition after being spread onto a subject's skin, the mold comprising 1.5% w/w of Agar-Agar; FIG. 5A presents a sunscreen molded composition after being lightly spread on skin (initial spreading);

FIG. 5B presents a fully spread and absorbed composition.

### DETAILED DESCRIPTION

The present disclosure is based on the development of a single dose formulation for topical application that has a solid yet deformable shape and is spreadable once sheared against a surface, such as a skin surface. The single-dose formulation comprises a combination of a lipophilic and hydrophilic material that forms together an emulsion gel which after molding retain its discrete shape at temperatures of even up to 50° C. It has been found that the resulting formulation while having a solid shape, can be easily and conveniently applied to the skin by even gentle shearing against the skin.

Specifically, the present disclosure provides, in accordance with a first of its aspects, a solid mold composition comprising an emulsion gel including a gel matrix, water, and lipophilic droplets dispersed within the gel matrix, the composition further comprising one or more emulsifiers and lipophilic material, the latter being present at a concentration of at least 5% by weight out of the total weight of the mold. The gel matrix is formed of at least one gel forming material that is water insoluble at a temperature below 40° C. and that is present in the mold at a concentration higher than 1% (w/w) out of the total weight of the mold. Further, 80% of the emulsifiers comprised in the composition are of a type known to be solid at room temperature.

The term “emulsion gel” as used herein relates to a class of soft solid-like gel of two types of structures: (1) a polymeric gel matrix into which emulsion droplets are integrated, which are also known as emulsion-filled gels, or (2) aggregated emulsion droplets in the form of a network, which are also known as emulsion particulate gels.

In connection with the above, and without being bound by theory, it is assumed that the molded composition disclosed herein is composed of an emulsion particulate gel, i.e. the 2^{nd} type of emulsion gel described above. According to Farjami and Madadlou (2019, ibid.), emulsion particulate gels are formed of clustering and gelation of emulsion droplets which form a continuous three-dimensional network. The emulsion droplets stick together in a reversible manner, by moderately week forces to form a transient network possessing viscoelastic properties of a weak gel-like solid.

In order for the solid molded composition to be formed and maintain its physical nature, i.e., the solid, discrete shape even when extracted from the casting structure or any other type of housing of the composition, and yet be able to deform/spread upon shearing against a surface, there is a use of a type of emulsifiers, a minimal amount of the gel-forming material, and a unique balance between the two.

In addition to the specific selection of emulsifiers and gel forming material, the solid molded compositions of the present disclosure comprise lipophilic material in an amount of at least 5% by weight out of the total weight of the composition. In some cases, the compositions comprise at least 7, 8, 9, 10, 11, 12, 13, 14 or 15% w/w of lipophilic material out of the total weight of the composition. Each is an embodiment of the herein disclosure. In some examples, the composition comprise at least 10% w/w lipophilic material.

In the context of the present disclosure, the term solid mold or solid mold composition should be understood as any structured mass that is formed by allowing the emulsion gel in a fluid state to solidify in a pre-defined spatial shape and that the shape is maintained even if the molded composition is extracted from its container/housing or from the physical cast in which the molded composition is formed. The molded composition disclosed herein should thus be distinguished from firm creams within their containers which, while retaining their shape while in the container, cannot be extracted from their container without change in their shape or being deformed. Compositions that do not fall within the scope of the present invention may also be of the kind that are fragile, i.e. once extracted from the cast, crack or break.

Maintaining the integrity of the molded composition once extracted from the cast and during storage is not obvious, particularly in view that the composition comprises a combination of gel forming material, such as agar (typically used for water-based formulations) lipophilic material that are composed of small lipophilic globulars that would weaken the water based, gel component.

In the context of the present disclosure, the terms gel-forming agent, gel-forming material, gelling agent, and solidifying agent, are synonyms and therefore are and can be used interchangeably herein.

It has now been found that small amounts of gel-forming material (1% w/w and below) would lead to a non-stable mold (structurally and/or physically unstable), where, for example, water may evaporate out of the molded composition, and/or the physical strength of the mold may be subordinated. It has been found that the syneresis of such system would be significantly higher.

Thus, it has been concluded that the molded composition should include more than 1% w/w gelling agent.

The at least one gel forming agent is at a concentration that, in combination with water and one or more emulsifiers of a type as defined herein, is sufficient to maintain a firm (solid) shape holding lipophilic droplets, and yet, form into a smooth spreadable fluid once pressure is applied thereon.

In some examples, the concentration of the at least one gel forming agent is above 1% by weight and equal or below 10% by weight out of the total weight of the molded composition.

In some other examples, the concentration of the at least one gel forming agent is above 1% and equal or below 5%. In some other examples, the concentration of the at least one gel forming agent is above 1% and below 2.5%. In some other examples, the concentration of the at least one gel forming agent is between 1.1% and 2.4%. In yet some other examples, the concentration of the at least one gel forming agent is between 1% and 2.3%. In some examples, the concentration of the at least one gel forming agent is 1.7%±0.5% by weight, out of the total weight of the molded composition.

In some other examples the concentration of the at least one gel forming agent is higher than 1%, at times at least 1.1%, at times at least 1.2%, at times at least 1.3%, at times at least 1.4%, at times at least 1.5%, at times at least 1.6%, at times at least 1.7%, at times at least 1.8%, at times at least 1.9%, at times at least 2%, at times at least 2.1%, at times at least 2.2%.

At times, the concentration is at most 4%, at times at most 3%, at times at most 2.9%, at times at most 2.8%, at times at most 2.7%, at times at most 2.6%, at times at most 2.5%, at times at most 2.4%, at times at most 2.3%, at times at most 2.2%, and at times at most 2.1%.

In some examples, the concentration of the gel forming agent(s) is within any combination of lower and upper limits listed above.

Without being bound thereto, it has been found that an amount of gel forming agent that is too high, e.g. above 10% would result in a fragile molded composition that cracks while being extracted from the case and/or being spread. Such a molded composition would exhibit granular and non-homogenous texture when spread.

Thus, the ability to maintain the appropriate range of the gel forming agent in the formulation is required to obtain a solid discrete molded composition which is homogenously spreadable on skin, without leaving emulsion or granular residues.

It has also been found that the combination of the type of emulsifiers as described herein (as being solid at room temperature) and the amount of the gel-forming materials as defined herein, is essential for the formation of the discrete bodies of solid molded composition disclosed herein, and preferably those having the strength and/or stability to be retained in a storage container (after being removed from its casting) in their discrete solid form for long periods of time without losing its integrity or weight (per mold unit), without changing its overall shape, and concurrently, maintaining its unique viscoelastic features so when a user extracts a discrete body from a container and applies it against the skin, the mold deforms smoothly so no particulate matter or solid leftovers remains on the skin.

Thus, the type of emulsifiers, i.e., being solid at room temperature, is also to be taken into consideration. Notably, when referring to room temperature it is to be understood as a temperature within the range of 18° C. and 28° C., typically, 25° C.±2° C.

Generally, the emulsifiers are of a type acceptable for human use and specifically, acceptable for use in topically applied formulations.

The emulsifiers (which may also be called surfactants) can be divided into groups: anionic, cationic, and non-ionic.

Molded composition of the present disclosure can comprise ionic emulsifiers only, non-ionic emulsifiers only, cationic emulsifiers only and combinations thereof. Each possible combination constituting a separate embodiment of the present disclosure.

In accordance with one example of the present disclosure, there is at least one non-ionic emulsifier in the molded composition. A non-limiting list of non-ionic emulsifiers that can be used in the mold disclosed herein includes, glyceryl stearate, Polysorbates (e.g., Polysorbate 20, 60, 80) polyethylene glycol, mono, and di glyceride as well as possible combinations of same.

At times, in order to obtain the disclosed solid molded composition, one should maintain a balance between ionic/non-ionic surfactants. the emulsifiers are further described below.

In one example, the at least one non-ionic emulsifier comprises any type of glyceryl stearate, PEG-distearate, (e.g., PEG-2 Distearate, PEG-3 Distearate, PEG-4 Distearate, PEG-6 Distearate, PEG-8 Distearate, PEG-9 Distearate, PEG-12 Distearate, PEG-20 Distearat, PEG-32 Distearate, PEG-75 Distearate, PEG-120 Distearate, PEG-150 Distearate, PEG-175 Distearate), and particularly, PEG-150 distearate.

In accordance with some other examples, there is at least one anionic/cationic emulsifier, such as Potassium cetyl phosphate and/or sodium lauryl sulfate.

The overall amount of emulsifiers in the molded composition may vary depending on the type and amount of gelling agents used. Yet, in some examples, the amount of the emulsifiers in the molded composition can range between about 2% by weight to about 10% by weight out of the total weight of the solid mold composition, at times, between 3% to about 8%, at times between 4% to 6% by weight, out of the total weight of the mold.

In some other examples, the amount of emulsifiers in the molded composition can be at least 4.5%, at times, at least 5%, at times, at least 6% by weight out of the total weight of the solid mold composition. At times, the amount is not more than 10%, at times, not more than 9%, at times, not more than 8%, at times, not more than 7%, at times, not more than 6% by weight, out of the total weight of the mold.

In some examples, the amount of emulsifiers in the molded composition can is between 4.5 and 8% w/w, at times between 4.5 and 7% w/w.

In some examples, the emulsifiers comprise any combination of fatty acids (e.g. palmitic acid, stearic acid) and/or fatty acid alcohols (e.g. stearyl alcohol), and/or glyceryl stearate and/or potassium cetyl phosphate.

In some examples, the emulsifiers can be selected according to their Hydrophilic-lipophilic balance (HLB). HLB is an acceptable measurement of the degree to which an emulsifier or a surfactant is hydrophilic or lipophilic. Generally, such a value is determined by calculating the values of different regions of the molecule.

There are at least two methods for determining an HLB value of a molecule (e.g. an emulsifier): (1) Griffin's method (mostly applicable for non-ionic surfactants): based on the molecular mass of the hydrophilic portion of a molecule and the molecular mass of the whole molecule; and (2) Davies' method: which is based on the chemical groups of a molecule, while taking into account the effects of stronger and weaker hydrophilic groups. One skilled in the art would know which method to use for determining the HLB, and thus the type of emulsifier to use in the context of the present disclosure.

In some examples, the emulsifiers which are solid at room temperature constitute at least 50% of the total amount of emulsifiers of the molded composition. At times, the amount of emulsifiers which are solid at room temperature are at least 60%, at times at least 70%, at times at least 75%, at times at least 80%, at times at least 85%, at times at least 90%, at times at least 95% out of the total amount of emulsifiers in the solid molded composition.

In some specific examples, the amount of the emulsifiers which are solid at room temperature out of the total amount of the emulsifiers of the mold is at least 80% w/w.

In some examples, emulsifiers which are solid at room temperature are selected from Stearic acid, Palmitic acid, Stearyl Alcohol, Potassium Cetyl Phosphate, Peg-150 Distearate, Glyceryl Stearate Citrate, Glyceryl Stearate, Glyceryl Stearate, PEG-100 Stearate, Polyglyceryl-3 Distearate, Glyceryl Stearate Citrate, Glyceryl Stearate Citrate, Cetearyl Alcohol, Glyceryl Caprylate, Polyglyceryl-3 Methylglucose Distearate, Cetearyl Glucoside, Methyl Glucose Sesquistearate, Polyglyceryl-3 Dicitrate/Stearate, Sucrose Stearate, Glycol Stearate SE, Glyceryl Stearate, Ceteareth-20, Ceteareth-25. Cetearyl Alcohol, Coco-Glucoside. Arachidyl Alcohol, Behenyl Alcohol, Arachidyl Glucoside and Cetearyl Alcohol, each emulsifier forming an independent embodiment, which may be independently combined with one or more of the other independent emulsifiers listed above, any combination constituting a separate embodiment of the present disclosure.

In some specific examples, emulsifiers which are solid at room temperature are selected amongst Stearic Acid, Palmitic Acid, Stearyl Alcohol, Potassium Cetyl Phosphate, Peg-150 Distearate and/or Glyceryl Stearate, and any combination of same.

In some other examples, the composition comprises also emulsifiers which are liquid at room temperature. Without being limited thereto, the emulsifier liquid at room temperature can be selected from the group consisting of Glyceryl Oleate Citrate, Polysorbate 80, Polysorbate 20, Sorbitan Laurate, Polyglyceryl-4 Laurate, Dilauryl Citrate, Sorbitan Tristearate, Sorbitan Oleate, PEG-7 Glyceryl Cocoate and PEG-40 Hydrogenated Castor Oil, and any combination of same, each possible combination constituting a separate embodiment of the present disclosure.

The gel-forming agent forms a gel when brought into contact with water. The amount of water (as well as other factors) can affect the viscoelastic properties of the gel thus formed. It has been found that for the desired performance (e.g., smooth and uniform application onto a skin) the weight ratio between the gel forming material and the water should be in a weight ratio between 1:25 and 1:55, at times, between 1:30 and 1:50, at times, between 1:25 and 1:40.

In accordance with some examples, the at least one gel forming agent is or comprises a polysaccharide. A non-limiting list of polysaccharides that can be utilized in accordance with the present disclosure includes Agar-Agar, carrageenan, Locust bean gum (L.B.G), Pectins, Alginate.

In some examples, the at least one gel forming agent is or comprises Agar-Agar. As appreciated, Agar-Agar is considered insoluble in water at temperatures below 90° C., although there are some preparations that hydrate and dissolve at about 70° C.−90° C.

Agar or agar-agar (which are synonyms in the herein context and can be used interchangeably) is a mixture of two main components: (1) agarose—which is a linear polysaccharide consisting of repeating units of agarobiose, which is a disaccharide consisting of D-galactose and 3,6-anhydro-L-galactopyranose, and (2) agaropectin—which is a heterogeneous mixture of smaller molecules, which mainly consists of D-glucuronic acid and pyruvic acid. Typically, Agarose constitutes about 60-70% of the mixture

In the field of formulations (skin care, cosmetic care, personal care, etc.) and pharmaceuticals Agar is known for its ability to form a gel. Thus, it is recognized as a gel-forming agent or a gelling agent. The gel-forming entity in agar is the unbranched, linear polysaccharide (agarose).

When defining agar in chemical terms, one of skill in the art would refer to it as a polymer comprising subunits of the sugar galactose.

The agaropectin in Agar mostly consists of alternating units of D-galactose and L-galactose which are modified with acidic side-groups, e.g., sulfate and pyruvate.

In some examples, the solid molded comprises an emulsion gel comprising a gel matrix, water, and lipophilic droplets comprising one or more emulsifiers and lipophilic material, the lipophilic droplets being dispersed within the gel matrix, wherein the gel matrix is formed of at least one gel-forming material, wherein the at least one gel forming material is agar;

wherein the agar is present at a concentration higher than 1.0% by weight out of the total weight of the solid mold;

wherein the lipophilic material is present at a concentration of at least 5% by weight out of the total weight of the solid molded composition;

wherein at least 80% of the emulsifiers are solid at room temperature; and

wherein the combination of said at least one gel-forming material, one or more emulsifiers, lipophilic material, and water provide said solid mold with a discrete shape that is deformable upon shearing against a surface.

In some examples, when the gel forming agent is agar, the agar comprises agarose at an amount between above 1%, at times between 1% to about 1.8% w/w out of the total weight of the solid molded composition.

In some examples, the amount of agarose is between about 1.2 to 1.4% w/w.

In one specific example, the amount of agarose out of the total amount of the composition is about 1.32% w/w.

In some examples, the agaropectin component of the agar comprises between about 3% to about 10% of ester sulfates.

In some examples, the agar is a Gold Agar - cosmetic grade agar—such as Agar from Hispanager (product code GAHSALNP0000), having the following properties (in comparison with a standard Agar):

| General Properties | Standard Agar | | Gold Agar Hispanagar | |
|---|---|---|---|---|
| Particle size | 100 | mesh | 60 | mesh |
| Moisture content | Max 18% | | Max 20% | |
| Water absorption | Max 75 ml | | Max 75 ml | |
| PH | 6.5-7.5 | | 6-7.5 | |
| Water gel strength | 700-1100 | g/cm² | 700 | g/cm² |
| Nikan method at 1.5% | | | | |
| at 20° C. | | | | |
| Setting point | 32-45° | C. | 34-38° | C. |
| Melting point | 85-95° | C. | 85-95° | C. |

| | | |
|---|---|---|
| AGAROSE % | 66.6 | 60-70 |

In some other examples, the at least one gel forming agent is or comprises carrageenan. Carrageenan is water insoluble at temperatures below 40° C.

In the context of the present disclosure, carrageenan is defined as a lineage of linear sulfated polysaccharides extracted from red seaweeds.

Molecules of carrageenan are mostly large (having high molecular weight), and highly flexible polysaccharides consisting of repeating units of galactose and 3,6 anhydrogalactose (3,6-AG) which may be sulfated or non-sulfated. The molecules typically form helical structures.

Three main classes of carrageenan are used in industry and these include:
1. Kappa—typically used to form strong and rigid gels;
2. Iota—typically used to form soft gels when combined with calcium ions; and
3. Lambda—typically used to thicken diary products.

Solid molded compositions comprising carrageenan as a gelling agent are sensitive to the amounts of electrolytes (mostly cations) therein. In examples where kappa carrageenan is being used, higher amounts of potassium ions would form a gel which is more rigid.

In examples where iota carrageenan is being used, increasing the amounts of calcium ions would result in a more rigid gel.

Concentrations of electrolytes (cations in most cases) would also determine the gelling temperature and the melting temperature of the formed gel in a way that increasing the amounts of the cations would increase the gelation point and the melting point.

In some cases, the gelation point would increase from about 35° C. to about 60° C. In some cases, from about 35° C. to about 65° C. In some cases, from about 35° C. to about 70° C. In some cases, from about 35° C. to about 75° C. In some cases, from about 35° C. to about 80° C.

A person of ordinary skill in the art would know to pick the appropriate type of carrageenan (kappa, iota or lambda) or combinations thereof and balance it with the right amount of electrolytes in order to arrive to a solid composition as described herein.

In some examples, the carrageenan is selected from kappa, iota, lambda and a combination thereof.

In some examples, the carrageenan is kappa carrageenan.

In some other examples, the carrageenan is iota carrageenan.

In yet other examples, the carrageenan is a combination of kappa and iota carrageenan.

In yet other examples, the carrageenan is a combination of kappa and iota carrageenan.

The amount of the lipophilic phase is also essential when choosing the type of carrageenan and the amounts of electrolytes. In examples where the lipophilic phase is in amounts higher than 5% w/w, the gelling agent may also comprise lambda carrageenan.

In one specific example, where kappa carrageenan is being utilized in an amount of 1.5% w/w, the appropriate amount of potassium ions to be added is about 0.2% w/w, to obtain a gel having a gel strength of between 500 to 1200 g/cm² in about 20° C. Same amounts of kappa carrageenan without the potassium cations would result in a gel having a reduced gel strength between about 100 to about 350 g/cm².

The solid molded composition can contain a single gel forming agent or a combination of gel forming agents. Thus, in the context of the present disclosure, when referring to at least one gel forming agent it is to be understood as referring to one or a combination of two, three, or more gel forming agents, at least one having poor water solubility.

In some examples, when the gel-forming material is agar-agar, the ratio of agarose:agaropectin can determine the strength of the gel, such that, a higher agarose:agaropectin ratio would result in a stronger and more rigid gel within the molded composition. The use of agar-agar is further discussed below.

In some other examples, the strength of the solid molded composition can be determined by the holding period (incubation period) of the polymers at temperatures of between 85° C.−95° C. Longer periods of incubation (or holding) when the pH is equal or below 6, would result in a weaker gel.

In yet some other examples, the pH can play an important factor in the strength of the gel, such that lower pH would result in a weaker gel and vice versa.

For example, the ability of agar (as an exemplary gel forming material) to maintain its stability and form a gel with limited syneresis (extraction of liquid from a gel) would be associated with hydration abilities of the agar and the electrical charge (such as that of the sulfates of agaropectin) which induces a repulsion between polymeric molecules. Reducing the amount of water and neutralizing the negative electric charges of agaropectin, for example, by lowering the pH (specifically below 6) would lead to flaking of the agar and to an unstable gel.

The mold composition can be formed by introducing the emulsion gel in a fluid state into a cast or onto a plate/surface and allowing the emulsion gel to cool therein or thereon, respectively. The placing of the emulsion gel into the cast or onto the plate can be continuous or in batches by any means known in the art and as further discussed below.

In some cases, the shaping into a predefined steady and stable physical form, i.e., into the shape of the solid mold, can be pouring the hot emulsion into a cold media. A unique feature of the molded composition disclosed herein is that once sheared against a surface, e.g., the bodily surface, the solid mold deforms into a smooth spreadable matter without breaking into smaller solid masses/clumps. Thus, while retaining its solid and discrete form at storage and even at temperatures reaching 50° C., pressing the solid form causes the shaped form to deform into a spreadable formulation.

Yet another unique feature of the molded composition is the irreversibility thereof after the emulsion is being molded. In other words, after being prepared via the methods described herein, molded compositions of the disclosure maintain their solid molded form even after heating them to temperatures equal or higher than 50° C., at times equal or higher than 60° C., at times equal or higher than 70° C., and at times equal or higher than 80° C.

The solid molded composition disclosed herein can be characterized by unique hardness, adhesiveness, cohesiveness, and elasticity, all contributing to the viscoelastic properties of the mold disclosed herein. These properties can be determined using a Texture Profile Analyzer such as the TA1 series Texture Analysis Machine (LLOYD instruments) using a cylindrical probe of 1.5 inches.

In some examples, the solid molded composition is characterized by hardness above 1 N/m², at times above 2 N/m², at times above 3 N/m², at times above 4 N/m², at times, above 5 N/m², or even above 6 N/m², when the probe is pressed at least 50% of its distance (pressure length) into the tested sample.

In some example, the solid molded composition is characterized by hardness above 16 N/m², at times above 20 N/m², at times above 25 N/m², at times above 30 N/m², at times, above 35 N/m², or even above 40 N/m², when the probe is pressed at least 85% of its distance (pressure length) into the tested sample.

In some example, the solid molded composition is characterized by adhesiveness above 4 N/mm, at times above 8 N/mm, at times above 12 N/mm, at times above 16 N/mm, at times, above 20 N/mm, or even above 25 N/mm, when the probe is pressed at least 85% of its distance (pressure length) into the tested sample.

In some examples, the elasticity of the solid molded composition, as determined by springiness index (the ratio of the height to which the sample springs back after the first bite relative to total compression distance of the bite) is above 0.4.

In some examples, the emulsion used for the molded composition has a viscosity of at least 1,000 centipoises at a temperature above 50° C. In some examples, the emulsion used for the mold has a viscosity of at least 5,000 centipoises at a temperature above 50° C. The viscosity is determined, in accordance with some examples, by Viscometer VT06 Rion Co. Ltd, spindle No.1.

The solid molded composition comprises an emulsion gel formed of one or more gel forming material. A gel forming material, also known and equivalently referred to herein as a gel forming agent or a gelling agent, is a material that alters the three-dimensional networks of the polymeric chains within the liquid, e.g. water, by forming into a cohesive matrix, namely, a gel.

The selection of the at least one gel forming agent is such that the mold is maintained structurally stable after being removed from its casting mold, during storage, and after extraction from the storage container (before being spread onto body portion), and in this context, storage means at any temperature of even up to 40° C. or even up to 50° C. or below even −10° C. To allow this stability, it has been envisaged that at least one of the gel forming agents, at times, the combination of the gel forming agents, are water insoluble at a temperature below 40° C., at times, at a temperature below 50° C. In other words, the at least one gel forming agent/material used in accordance with the present disclosure is one that becomes soluble upon heating to a temperature above 40° C., at times, above 50° C., at times above 60° C., at times above 70° C., at times above 80° C. The solubility of gel forming agents in known in the art or can be determined/verified by any standard water solubility test known in the art.

Without being bound thereto, it is envisaged that the gel-forming agent mostly determines the strength of the molded composition, while the type of emulsifiers defines the stability of the solid form of the molded composition. For instance, employment of a significant amount of emulsifiers which are liquid at room temperature or in other words, when less than 50% of the emulsifiers employed are solid at room temperature, or more than 50% of the employed emulsifiers, and/or at times also of the employed emollients are liquid at room temperature, may lead to weakening or even loss of the integrity of the molded shape. Such instability may specifically be manifested in spreading of material from the molded composition, water evaporation and/or increase in the syneresis of the solid molded composition and phase separation.

The gel forming material and the water (together with other materials of the compositions which are water soluble, such as and without limitation chelating agents or some emollients which are water soluble) define together a hydrophilic phase in the process of forming the emulsion gel of the mold as further discussed below.

The molded composition disclosed herein also comprises lipophilic components that together constitute a lipophilic phase in the process of preparing the molded composition, as further discussed below.

In the context of the present disclosure, the term lipophilic material encompasses a variety of compounds, e.g., organic compounds, with different functionalities, including emulsifiers, film-forming agents, thickeners, emollients, oils (e.g. plant based) etc. In some examples, the lipophilic material encompasses at least the said emulsifiers. In some other examples, the lipophilic material encompasses the said emulsifiers and emollients.

As noted herein, an important feature of the herein disclosed composition, which allows it to be in a solid discrete form, even when extracted from its casting mold, yet allows it to be deformable and spreadable in a consistent and convenient manner without leaving any solid leftovers on the bodily portion (i.e., viscoelastic characteristics), is the unique combination of emulsifiers which are solid at room temperature and a gel-forming material, in an amount which is higher than 1% w/w out of the total weight of the mold.

Yet, the composition can comprise other ingredients.

In some examples, the molded composition comprises at least polyethylene glycol (PEG) or derivatives thereof.

In some examples, the molded composition comprises PEG-150 Distearate. In some examples, the PEG-150 Distearate is an important element of the composition. In some examples, the PEG-150 Distearate is in an amount ranging from 0.01% to 1% by weight of PEG-150 Distearate, at times, 0.2-0.5%.

In some examples, the molded composition comprises one or combination of emollients. An emollient is typically used as a moisturizing agent that is able to provide a softening/smoothing effect on the skin without actually adding any moisture to the skin.

Emollients can be categorized into two types, oil-based, and water-based emollients/humecants). In accordance with the present disclosure, the product comprises at least oil based emollients. Such emollients form part of the lipophilic phase in the process of preparing the mold of the present disclosure.

In some examples the composition includes water based/hydrophilic emollients selected from glycerin, sorbitol, propylene glycol, propanediol, butylene glycol, Panthenol and any possible combination of same, each possible combination constituting a different embodiment.

Non-limiting examples of emollients include dicaprylyl carbonate, C12-C15 alkyl benzoate, octyl palmitate, dicaprylyl ether, coco caprylate, dibutyl adipate, isopropyl myristate, oleyl eurocate, caprylic/capric triglycerides, octyldodecanol, dipropylheptyl carbonate, vegetable oils, mineral oil, each emollient forming an independent embodiment, which may be independently combined with one or more of the other independent emollients listed above, any combination constituting a separate embodiment of the present disclosure.

In some more specific examples, the emollients include any one or combination of C12-C15 alkyl benzoate, coco caprylate, Dibutyl Adipate, each possible each possible combination constituting a different embodiment of the present disclosure.

In some examples, the molded composition comprises film forming agents. Generally, film forming agents used in the mold can include polyvinylpyrrolidone (PVP), acrylates, acrylamides, methacrylates, polyethylene and various copolymers. They typically have water-binding properties and are used to provide a pliable, cohesive and continuous covering over the bodily portion and to further increase water resistance.

A non-limiting list of film forming agents include, VP/Eicosene Copolymer, Acrylates/Octylacrylamide Copolymer, Styrene/Acrylates Copolymer, Polyurethane-34, Acrylates/C12-22 Alkylmethacrylate Copolymer, polyethylene, Polyurethane-64.

In some examples, the molded composition disclosed herein also comprises one or more humectants. Humectants are substances that are able to bond with water molecule from the environment, thereby increasing moisture content of the skin onto which the product is applied. There are different types of humectants, such as glycerin, sorbitol, propanediol, panthenol, hyaluronic acid, alpha hydroxy acids, salicylic acid and some sugars. Each of these humectants form an independent embodiment, which may be independently combined with one or more of the other independent humectants listed above, any combination constituting a separate embodiment of the present disclosure.

In some examples, the mold comprises at least glycerin.

In some specific examples, the humectants are selected from propanediol and panthenol and combinations thereof, combined in some examples with glycerin.

In preparation, the humectant can be added as part of the lipophilic phase or hydrophilic phase, or after the emulsion gel is formed, as further discussed below.

In a specific example, the humectants are part of the hydrophilic phase.

The molded composition disclosed herein can include additional additives, such as sunscreen agents, skin whitening agent, antibiotics, non-steroidal anti-inflammatory drugs (NSAID), anti-fungal, acne treatments, cannabidiols (CBD), preservatives, antioxidants, fragrances, colorants as known in the art.

When the molded composition is used for sun screening, this may affect the ratio between ingredients within the solid molded composition. A person skilled in the art of formulations (such as cosmetics, skin care and personal care) would know to balance the amounts and ratio.

Table 1 provides a non-limiting, exemplary lists of functional components in molds with or without sunscreen agents, both of the types disclosed herein, and their exemplary ranges of amounts.

TABLE 1
Functional components in sunscreen protecting mold

| | | Topical use |
|---|---|---|
| | Sun screen protection | (without sun-screen) |
| Functional ingredient | % by weight | |
| Water | 40%-60% | 40-90% |
| Sun screen agents | 5-45% | — |
| Gel forming material | <1.0% and up to 10% | <1.0% and up to 10% |
| Emulsifiers | 0-10% | 0-10% |
| Emollients | 2%-8% | 2-50% |
| pH adjusting agent | Any amount to reach pH 5.0-7.5 | |
| Film forming agent | 0.5%-10% | 0-10% |
| Humectants | 0.01%-50% | 0.01-50% |
| Chelating agent | Depends on gel forming material | |
| Preservatives | 0.01%-5% | 0.01%-5% |
| Other additives* | 0.5%-5% | 0.5-5% |
| Lipophilic phase | Add 100% | Add 100% |

*antioxidants, fragrances, colorants, actives, etc.

The molded composition disclosed herein, can also be characterized by the lipophilic droplets within the gel matrix holding the same, e.g., their desired average size range and/or desired size distribution. The average size and/or size distribution can be dictated by the manner of forming the emulsion gel.

In some examples, the average size of the droplets is below at times, below 900 μm, at times, below 800 μm; at times, in the range of 300 μm-800 μm; at times, in the range of 400 μm-700 μm.

In some examples, the size distribution of the droplets is a narrow size distribution. When referring to a narrow size distribution it is to be understood as one having a normal Gaussian/Bell curve with a deviation from the average of no more than 20%, at times 10%, this being equivalent to a standard deviation of SD20, at times, of SD15 or even of SD10.

The molded composition is prepared by using principles of homogenization. Specifically, the method comprises mixing a hydrophilic phase that comprises water, at least one gel forming agent and optionally a chelating agent, with a lipophilic phase comprising lipophilic material and one or more emulsifiers. The mixing is while the hydrophilic phase is at a temperature of at least 60° C., at times, at least 70° C., at times, at least 80° C. and the lipophilic phase is, independently at a temperature of at least 60° C. at times, at least 70° C., at times, at least 80° C. The mixing is until a homogenized emulsion gel is formed.

In accordance with some examples, the homogenization of the two phases is in a high-speed homogenizer with speed in the range of about 150-5,000 rpm.

The homogenized emulsion gel may then be treated, (if necessary) to adjust the pH of the emulsion gel to a pH in the range of 6-8, at times, in the range of 6.5 to 8, at times, in the range of 7-7.5. the liquid emulsion gel is then allowed to cool in a form of a mold.

The pH of the emulsion gel is adjusted by the addition of pH adjusting agents commonly used in the cosmetic industry. Without being limited thereto, the pH adjustment can be by using NaOH, Triethanolamine, KOH, Sodium Bicarbonate, Citric acid. The pH adjusting agent can be added to the hydrophilic phase, and/or to the combination of the two phases, each pH adjusting agent forming an independent embodiment, which may be independently combined with one or more of the other independent agents listed above, any combination constituting a separate embodiment of the present disclosure.

In some specific examples, the pH adjusting agent comprises at least NaOH. In some other specific examples, the pH adjusting agent comprises at least Citric acid.

As noted above, the cooling into a form of a molded composition is not necessarily via the use of a casting model, and cooling can also be in the form of sheets of the emulsion gel on a surface e.g., flat, or non-flat plate or tray and then sizing the laminate/sheet to the desired dimension for use as a single dose to be applied onto the bodily surface, e.g., skin.

The cooling of the emulsion gel is to a temperature that still maintains the emulsion gel in a fluid state. This allows the addition, in this state of any additional components, such as those that may be sensitive to the high temperatures at which the emulsion gel is formed. The addition of any other ingredients (see hereinabove) is typically by a homogenization process and/or stirring. In some examples, the introduction of additives is under stirring and while the temperature of the emulsion gel is maintained above 50° C.

After all desired or required ingredients is added, the final emulsion gel composition is placed on the molding shape/cast element and the emulsion gel is allowed to solidify by cooling.

Cooling can be at room temperature or by placing the emulsion gel in a cooling chamber or transferring the emulsion gel via a cooling chamber. In some examples, the cooling is to reach a core temperature of the mold of about 25° C.±3° C. or lower, e.g., even to a core temperature of 10° C.+3° C.

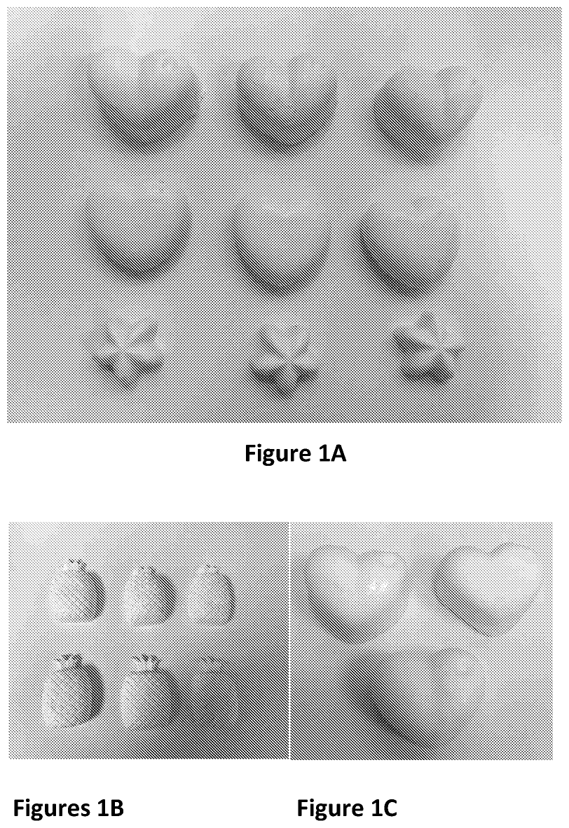

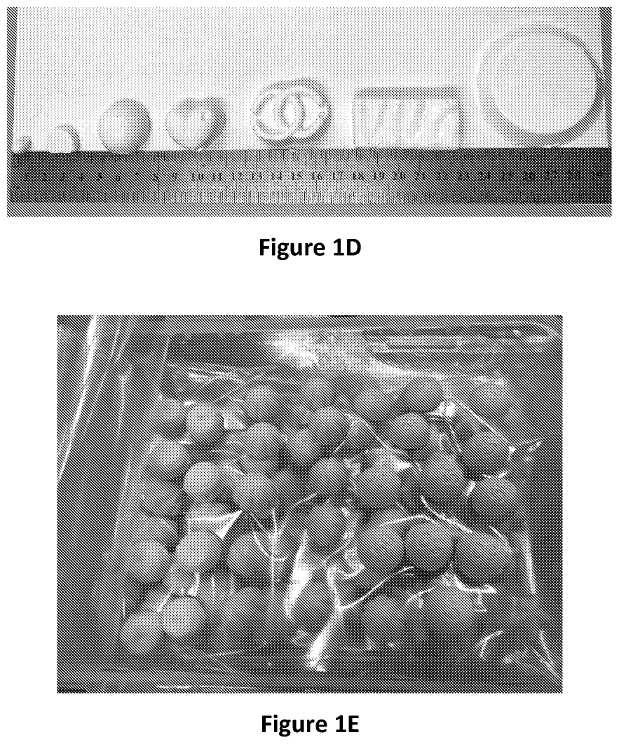

The molded composition disclosed herein can have any shape and size. It can have a define geometrical shape or an amorphous shape, it can have a 3D shape or a 2D shape, e.g., a sheet and the present disclosure should not be limited to the shape or size of the mold. FIGS. 1A-1E provide examples of different formulations, in different shapes, sizes and colors of molds in accordance with the present disclosure. Irrespective of the shape given to the molded composition, it is maintained as is, even when removed/extracted from the location at which it has received its shape (the casting structure and the like) or the location where it is stored (e.g., the storage container).

Further, after shaping (mold formation), the molded composition can be coated with additional coatings, e.g., by spray coating or other techniques. The coating may be for esthetic considerations, such as providing the mold with shininess, or for protection. The coating, however, would not affect the spreadable behavior (“spreadability”) or the stability and ability of the solid mold to maintain its shape and structure.

Each discrete solid molded composition can then be packed. Each package can include a single solid molded composition or a plurality of such discrete and separable molds (see FIG. 1E). When packed together, the plurality of molds are maintained as discrete bodies such that each can be picked separately without causing shape deformation or damaging the integrity of the shaped composition.

In fact, it has been found that the molded compositions do not stick to each other and maintain their spatial shape during storage.

In some cases, each molded composition is packed separately.

The package can be of any type, material or form and can contain any volume or weight of the mold depending on the desired dose to be applied onto the skin.

Upon use, the molded composition is lifted out of the package and sheared against the skin, until full coverage of the skin area. It has been found that all the mold-forming material is absorb or spread onto the skin without leaving any residue that needs to be cleared or discarded. In this respect, there is thus also provided herein a method comprising shearing one or more discrete molds disclosed herein against a body portion until mold-forming material is spread onto said body portion. In this connection, it is noted that the molded composition can be applied onto any needed bodily portion, such as the skin (including scalp), hair or even onto mucosal membrane. In one preferred embodiment, the method comprises applying the molded composition onto a skin portion.

The molded composition can be used for various applications, depending, inter alia, on the type of additives included therein. For example, if the additives include sun-screen agents/filters, the care mold can be used for sun protection.

Unless otherwise indicated, all numbers expressing quantities of ingredients and concentrations used herein should be understood as modified in all instances by the term “about.” The term “about” when used in connection with percentages can mean±1%. When used in connection with other measurements (such as temperature), the value can be ±10% of the original term (thereby, as an instance if the originally indicated temperature is about 60° C., that means that the temperature is actually ranging between 54-66° C.).

### NON-LIMITING EXAMPLES

### Example 1: Preparing Spreadable Mold of Sunscreen Formulation

The process of preparation included separate preparation of a lipophilic phase, a water/hydrophilic phase and combination of the two phases to form an emulsion.

### Preparing the Lipophilic Phase

The composition of the lipophilic phase is detailed in Table 2

TABLE 2
Lipophilic phase

| Ingredient | Function | % |
|---|---|---|
| Total sunscreen filters | | 30% |
| Octocrylene | Sun filter | 5-10 |
| Homosalate | Sun filter | 5-15 |
| Butyl Methoxydibenozylmethane | Sun filter | 2-5 |
| Ethylhexyl Salicylate | Sun filter | 2-5 |
| Total Emollients | | 6% |
| Dicaprylyl Carbonate | Emollient | 1-5 |
| C12-15 Alkyl Benzoate | Emollient | 1-5 |
| Total emulsifying agent | | 4.5% |
| Palmitic Acid, Stearic Acid | Emulsifying agent | 1-4 |
| Glyceryl Stearate | Emulsifying agent | 2-4 |
| Stearyl Alcohol | Emulsifying agent | 1-3 |
| Potassium Cetyl Phosphate | Emulsifying agent | 0.5-3 |
| PEG-150 Distearate | Emulsifying Agent | 0.1-0.5 |
| Total Film forming agent | | 1-2 |
| Polyethylene | Film forming agent | 1-2 |

The lipophilic raw materials as detailed in Table 1 were weigh and added into the tank, where they were heated to 80° C. while stirring. until homogenization. Preparing the hydrophilic phase

The composition of the hydrophilic phase is detailed in Table 3

TABLE 3
hydrophilic phase

| Ingredient | Function | % |
|---|---|---|
| Water (Aqua) | Solvent | 45-50 |
| AGAR-AGAR | Gelling agent | above 1 and less |
| | | than 2.5 |
| Disodium EDTA | Chelating agent | 0.05-0.5 |

The preparation of the hydrophilic phase is carried out in a double jacketed mixing tank (for optimum heat). Initially, disodium EDTA is dissolved in water, at room temperature, and once completely dissolved, AGAR-AGAR is added at high speed stirring and homogenization. As a final stage, the stirred dispersion is heated to 90° C.

### Mixing and Homogenization of the Two Phases

The lipophilic phase (kept at 80° C.) was slowly added into to the water phase (kept at 90° C.), while stirring and using a high-speed homogenizer until a first stage emulsion is formed. To the first stage emulsion, NaOH solution (solution at a temperature of above 45° C.) was added to form a pH adjusted first stage emulsion.

The pH adjusted first stage emulsion was then cooled in the tank to 65-60° C. and the pH of the emulsion was verified. If necessary, the pH can be corrected to be in the range of 7-7.5.

To further verify the formation of lipophilic droplets, the pH adjusted first stage emulsion was viewed and verified under a microscope (data not shown).

To the pH adjusted first stage emulsion, heat sensitive additives were added. The heat sensitive ingredients include preservatives, antioxidants, emollients, fragrances, and other additives, as detailed in Table 4.

TABLE 4
Heat sensitive additives

| Ingredient | Function | % |
|---|---|---|
| Total Preservatives | | 1.5 |
| Phenoxyethanol | Preservative | 0.5-1 |
| Ethylhexylglycerin | Preservative | |
| Chlorphenesin | Preservative | 0.2-0.4 |
| Phenylpropanol | Preservative | 0.01-0.3 |
| Caprylyl Glycol | Preservative | 0.01-0.3 |
| Other Additives | | 2-3 |
| Propanediol | Humectant | 0.5-1 |
| Perfume | Fragrance | 0.2-0.8 |
| Tocopheryl Acetate | Anti-oxidant | 0.1-0.5 |
| Camellia Sinensis | Anti-oxidant | 0.05-0.5 |
| Leaf Extract | | |
| Panthenol | Humectant | 0.05-0.2 |

The resulting emulsion gel including the heat sensitive additives, was then viewed under microscope to verify that no solidification appeared around small points of the nucleus which could affect the emulsion's uniformity (data not shown).

### Shaping by Molding

While kept at a temperature of about 60° C.-65° C., the liquid emulsion was poured into molds and immediately passed, at high speed, via a cooling chamber having an internal temperature set to 5° C.

FIGS. 1A-1B are images of solid molds comprising the emulsion gel obtained by the above example, FIG. 1A showing molds of the same shape and dimension, including different colorants, and FIG. 1B showing molds of different shapes and dimensions.

### Characterization:

### Texture Analyzer Test (Texture Profile Analyzer TPA)

The TPA method is a powerful tool that can provide highly meaningful insights into product texture. The method respects the fact that the textural identity of a product is both multi-faceted and inherently tied to consumers' sensory expectations. The advantage of TPA as an analytical method is that it can easily quantify multiple textural parameters.

In the following a TA1 series Texture Analysis Machine (LLOYD instruments, AMETEK, Manual: https://www.ametektest.com/-/media/ametektest/download_links/texture_analyzers_ta1_manual_english.pdf) was used to define rheological and structural characteristics of the gel-emulsion exemplified herein.

The TPA provides highly insights into product texture quantify multiple textural parameters. In the following examples, the LLOYD TA1 instrument was used and the collected data is presented in Table 5. specifically, the data concerns the following parameters:
Hardness-the resistance of a material to deformation of an indenter of specific size and shape under a known load.
Cohesiveness-the property of like molecules (of the same substance) to stick to each other due to mutual attraction.
Adhesiveness-the property of different molecules or surfaces to cling to each other
Springiness-Springiness was initially called “Elasticity” Springiness is how well a product physically springs back after it has been deformed during the first compression and has been allowed to wait for the target wait time between strokes. The springback is measured at the down-stroke.

All tests were conducted at room temperature, using a cylinder probe with a diameter of 1.5 inch.

The sample composition included the Mold exemplified herein. This was compared to two commercially available “Market” products [night cream in the form of pearls, from Payot (“p”), https://capelino.com/wanted-night-cream-in-the-form-of pearls-and-black-magnetic-mask-from-payot/] and J. ONE HANA (“j 1”) anti-wrinkle balls.

The results are presented in Table 5:

TABLE 5
TPA analyzer of the Mold exemplified herein
in comparison with two commercial products

| | | | Market | Market | Market |
|---|---|---|---|---|---|
| Sample | Mold | Mold | (p) | (p) | (j1) |
| Weight (gr) | 4.75 | 4.75 | 0.6 | 0.6 | 4.2 |
| Pressure length | 50% | 85% | 50% | 85% | 85% |
| Hardness | 9.7 | 41 | 1 | 6 | 18 |
| (N/m²) Bite 1 | | | | | |
| Hardness | 6.6 | 43 | 0.8 | 16 | 8 |
| (N/m²) Bite 2 | | | | | |
| Adhesiveness | −4 | 28 | −2 | 0.2 | 3.3 |
| N/mm | | | | | |
| Cohesiveness | 0.33 | 0.55 | 0.2 | 0.6 | −0.003 |
| Spreadability | N/A | 4.5 | | 4.5 | N/A |
| DIA (cm) | | | | | |
| Springiness | 0.6 | 0.5 | 0.6 | 0.4 | 0.5 |
| Index | | | | | |

The data presented in Table 5 provides the following observations.
The molded composition (Mold) exemplified herein has a greater hardness in all pressure lengths measured, being above 6 (N/m²) with 50% pressure length and above 40 (N/m²) with 85% pressure length. This hardness contributes to the structural stability/structure integrity during storage of the mold. From this we can conclude that the hardness of the mold disclosed herein should be above 1.
The molded composition (Mold) exemplified herein has adhesiveness high adhesiveness, of above 25 N/mm, when measured using a pressure length of 85%. The adhesiveness is also a feature that contributes to the stability of the mold. From this we can conclude that the adhesiveness of the mold disclosed herein should be above 4 N/mm.
The cohesiveness of the molded composition (Mold) was higher than that of the market product of essentially the same weight (4.2 gr), again, a characteristic contributing to the structural stability (integrity) of the mold.
The determination of elasticity (springiness) showed a ratio of 0.5-0.6 in all the samples (Mold and market products), indicative that the Mold meets the viscoelasticity requirements.

Without being bound to the following, the results support the statement with respect to the stability during storage of the molds disclosed herein, handling and ease of spreading under typical hand pressure.

In theory, it can be understood that the attraction forces between the lipophilic and hydrophilic molecules are strong and the emulsion forming the mold is very stable compared to market samples. The force necessary to overcome the attractive forces between the surface of the product and the surface of the skin allowed it to spread evenly.

Therefore, the mold disclosed herein spread uniformly without phase separation compared to market samples.

The results disclosed herein are in line with skin impression test when the product was applied onto the skin.

### Example 2: Preparing Spreadable Mold Hand Cream

The process of preparation of a hand cream mold (that does not include sun screen filters) was essentially similar to that performed above. The final formulation of Table 6 was obtained:

TABLE 6
Hand Cream Formulation

| Phase | Ingredient | Function | % |
|---|---|---|---|
| Hydrophilic | Water | Solvent | 70-75 |
| Phase | Agar | Gelling agent | 1-2 |
| | Disodium EDTA | Chelating Agent | 0.05-0.2 |
| Lipophilic | Ethylhexyl Palmitate | Emollient | 4-6 |
| Phase | Ceteary Alcohol, | Emulsifier | 3-5 |
| | PEG-20 Stearate | | |
| | Dimethicone | Skin Conditioning | 0.5-1 |
| | | Agent | |
| | Cetyl Alcohol | Co-emulsifier | 3-5 |
| | Cetearyl Alcohol, | Emulsifier | 3-5 |
| | Sodium Cetearyl | | |
| | Sulfate | | |
| | PEG-150 Distearate | Viscosity Controller | 0.01-0.5 |
| | Mangifera Indica | Skin Conditioning | 0.1-0.5 |
| | (Mango) Seed Butter | Agent | |
| Additives | Sodium Hydroxide | pH Controller | 0.01-0.1 |
| | Propylene Glycol | Humectant | 1-3 |
| | Glycerin | Humectant | 2-4 |
| | Hamamelis Virginiana | Skin Conditioning | 1-3 |
| | (Witch Hazel) Leaf | Agent | |
| | Water | | |
| | DMDM Hydantoin | Preservative | 0.2-0.4 |
| | Phenoxyethanol | Preservative | 0.5-1 |
| | Ethylhexylglycerin | | |
| | Tocopheryl Acetate | Antioxidant | 0.1-0.5 |
| | Panthenol | Skin Conditioning | 0.1-0.3 |
| | | Agent | |
| | Parfum (Fragrance) | Parfum | 0.2-0.8 |

### Example 3: Effect of Alteration of Agar-Agar Concentration on the Mechanical Properties of the Molded Composition

The following Table 7 provides examples within and outside the range of the gel forming material as described in the present disclosure. The only parameter which is altered is the concentration of agar-agar, where the rest of the ingredients are as portrayed throughout the non-limiting Examples (Examples 1-2).

TABLE 7
Agar concentrations

| | |
|---|---|
| Property | Description |

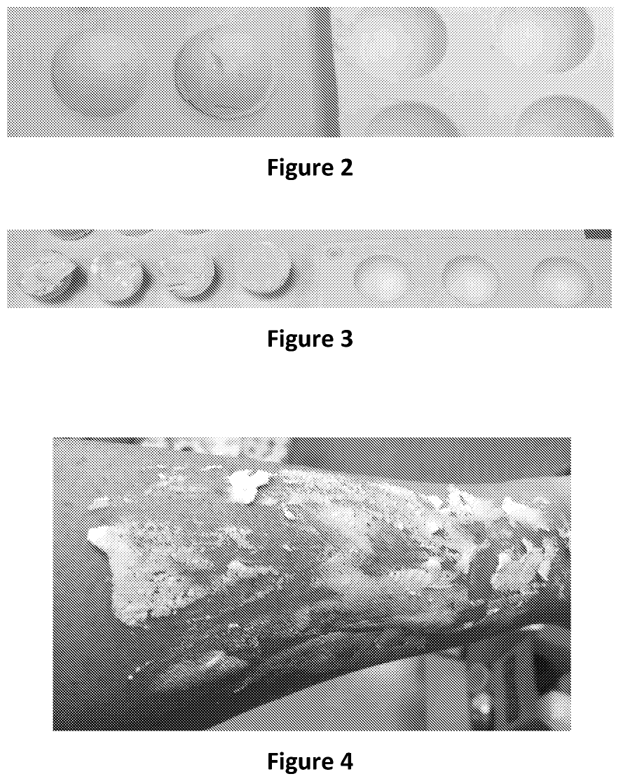

1% Agar-Agar composition (sun-screens)

| | |
|---|---|
| Release from | Was sensitive to release from casting mold and the |
| cast | integrity of the mold was damaged |
| Texture | very soft and any slightest pressure damaged the |
| | integrity of the structure of the molded composition. |
| | In addition, the composition left emulsion residues on |
| | skin upon spreading |
| Durability test | During stability test, the water evaporation rate high, |
| | with specific increase at accelerated temperature of |
| | 40° C. and 50° C. |
| Representative | FIG. 2 (molded composition) |
| image | |

1.2% Agar-Agar composition (sun-screens)

| | |
|---|---|
| Release from | Easily extracted, without any significant damage to |
| cast | structure |
| Texture | Solid and discrete; deformed and easily spread when |
| | applied to skin |
| Durability test | Water evaporation rate was slightly observed at an |
| | accelerated temperature of 40° C. and 50° C. |
| Representative | FIG. 3 (molded composition) |
| image | |

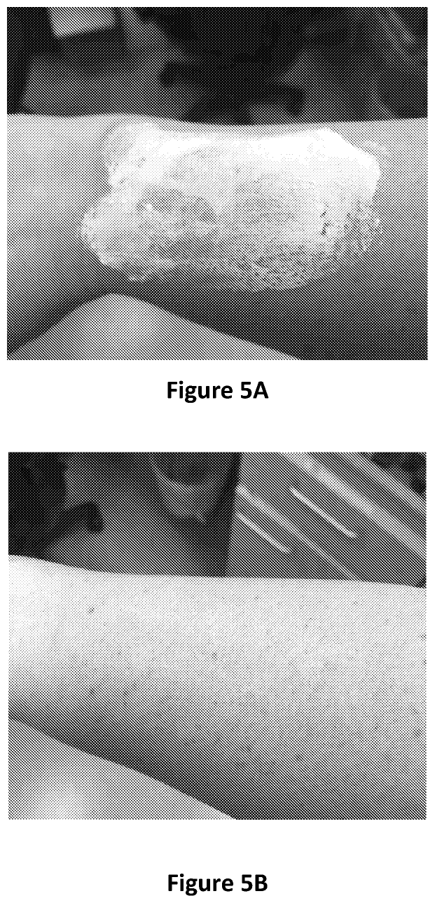

1.5% Agar-Agar composition (sun-screens)

| | |
|---|---|
| Release from | Easily extracted, without any significant damage to |
| cast | structure |
| Texture | Solid and discrete; homogenously and easily spread |
| | when applied to skin |
| Durability test | Stable at an accelerated temperature of 40° C. |
| | and 50° C. |
| Representative | FIGS. 1A-1E (molded compositions) |
| image | FIGS. 5A-5B (composition spread on skin) |

2.0% Agar-Agar composition (sun-screens and hand cream)

| | |
|---|---|
| Release from | Easily extracted, without any significant damage to |
| cast | structure |
| Texture | Solid and discrete; homogenously and easily spread |
| | when applied to skin |
| Durability test | Stable at an accelerated temperature of 40° C. |
| | and 50° C. |
| Representative | FIGS. 1B (sun-screen) and FIG. 1C (hand cream) |
| image | |

2.5% Agar-Agar composition (sun-screens)

| | |
|---|---|
| Release from | Easily extracted, without any significant damage to |
| cast | structure |
| Texture | When applying to the skin of the subject, the mold |
| | cracked (did not spread smoothly on skin). The appear- |
| | ance of the spread was granular and non-homogenous |
| Durability test | Stable at an accelerated temperature of 40° C. and |
| | 50° C. |
| Representative | FIG. 4 (non-homogenous spread of composition on skin) |
| image | |

1. A solid molded formulation comprising an emulsion gel comprising a gel matrix, water and lipophilic droplets comprising one or more emulsifiers and lipophilic material, the lipophilic droplets being dispersed within the gel matrix,
wherein
the gel matrix is formed of at least one gel forming material that is water insoluble at a temperature below 40° C.;
the at least one gel forming material is present at a concentration higher than 1% by weight out of the total weight of the solid molded composition;
the lipophilic material is present at a concentration of at least 5% by weight out of the total weight of the solid molded composition;
at least 80% of the emulsifiers are solid at room temperature; and
the said at least one gel forming material, one or more emulsifiers, lipophilic material and water provide together said solid molded composition with a discrete shape that is deformable upon shearing against a surface.

2. The solid molded composition of claim 1, in the form of emulsion particulate gel.

3. The solid molded composition of claim 1, wherein the at least one gel forming material comprises or is a polysaccharide.

4. The solid molded composition of claim 1, wherein the at least one gel forming material is selected from the group consisting of agar, carrageenan, Locust bean gum (L.B.G), Pectins and Alginate.

5. The solid molded composition of claim 4, wherein said at least one gel forming material comprises agar-agar.

6. The solid molded composition of claim 1, wherein the at least one gel forming material is at a concentration higher than 1.2% by weight and up to 10% by weight out of the total weight of the mold.

7. (canceled)

8. The solid molded composition of claim 5, wherein the agar-agar comprising agarose and agaropectin.

9. The solid molded composition of claim 8, comprising agarose in an amount of above 1% and up to 1.8% by weight, out of the total weight of the molded composition.

10.-11. (canceled)

12. The solid molded composition of claim 1, comprising at least 5% lipophilic material.

13. The solid molded composition according to claim 1 wherein the emulsifiers are present in the amount of at least 2% by weight out of the total weight of the molded composition.

14. (canceled)

15. The solid molded composition of claim 1, wherein said one or more emulsifiers comprise fatty acids.

16. The solid molded composition of claim 1, having a pH of between 5-7.5.

17. The solid molded composition of claim 1, being viscoelastic.

18. The solid molded composition of claim 1, having a gel forming material that has a viscosity of at least 1,000 centipoise at temperature above 50° C.

19. (canceled)

20. A method for preparing a solid molded composition, the method comprising:
mixing a hydrophilic phase that comprises water, at least one gel forming material and optionally a chelating agent, with a lipophilic phase comprising lipophilic material and one or more emulsifiers, said mixing is while said hydrophilic phase is at a temperature of at least 70° C. and said lipophilic phase is, independently, at a temperature of at least 60° C., until a homogenized emulsion gel is formed;
adjusting pH of the emulsion gel; and
molding the emulsion gel;
wherein the at least one gel forming material that is water insoluble at a temperature below 40° C.;
the at least one gel forming material is in an amount to provide a concentration higher than 1% by weight out of the total weight of the solid molded composition;
the lipophilic material is in an amount to provide a concentration of at least 5% by weight out of the total weight of the solid molded composition;
at least 80% of the one or more emulsifiers are solid at room temperature; and
the said at least one gel forming material, one or more emulsifiers, lipophilic material and water provide together a solid molded composition with a discrete shape that is deformable upon shearing against a surface.

21. (canceled)

22. The method of claim 20, comprising any one or combination of
homogenization of the hydrophilic phase prior to introducing thereto the lipophilic phase;
homogenization of the lipophilic phase prior to introducing the same into the hydrophilic phase:
adjusting of pH is to a pH in a range of 6.5-8;
cooling the emulsion gel to a temperature of between 80° C. to 50° C. before molding.

23.-25. (canceled)

26. The method of claim 20, comprising cooling the emulsion gel to a temperature of between 80° C. to 50° C. before molding and introducing into the cooled emulsion gel one or more heat sensitive additives, said introducing is under stirring and while maintaining temperature of the emulsion gel above 50° C.

27. (canceled)

28. A package comprising one or a plurality of discrete solid molds according to claim 1.

29.-30. (canceled)

31. A method of treatment comprising shearing against a bodily portion one or more solid molds according to claim 1.

32. A solid molded composition comprising an emulsion gel comprising a gel matrix, water and lipophilic droplets comprising one or more emulsifiers and lipophilic material, the lipophilic droplets being dispersed within the gel matrix,
wherein
the gel matrix is formed of gel forming material comprising at least agarose;
said agarose is present at an amount of above 1% by weight out of the total amount of the solid molded composition;
the lipophilic material is present at a concentration of at least 5% by weight out of the total weight of the solid molded composition;
at least 80% of the one or more emulsifiers are solid at room temperature; and
the said at least one gel forming material, one or more emulsifiers, lipophilic material and water provide together said solid molded composition with a discrete shape that is deformable upon shearing against a surface.
